SEQUENCE LISTING (9 sequences)

SEQ ID NO: 1 - DNA, 10 bp, Artificial Sequence
  Synthetic Polynucleotide
  misc_feature (1)..(10): Modified with a phosphorothioate linkage
  misc_feature (10)..(10): Modified with /iSp18//iSp18//branch//Stearyl// Stearyl/
tttttttttt

SEQ ID NO: 2 - DNA, 15 bp, Artificial Sequence
  Synthetic Polynucleotide
  misc_feature (1)..(15): Modified with a phosphorothioate linkage
  misc_feature (15)..(15): Modified with /iSp18//iSp18//branch//Stearyl// Stearyl/
ttttttttttttttt

SEQ ID NO: 3 - DNA, 20 bp, Artificial Sequence
  Synthetic Polynucleotide
  misc_feature (1)..(20): Modified with a phosphorothioate linkage
  misc_feature (20)..(20): Modified with /iSp18//iSp18//branch//Stearyl// Stearyl/
tttttttttttttttttttt

SEQ ID NO: 4 - DNA, 25 bp, Artificial Sequence
  Synthetic Polynucleotide
  misc_feature (1)..(25): Modified with a phosphorothioate linkage
  misc_feature (25)..(25): Modified with /iSp18//iSp18//branch//Stearyl// Stearyl/
ttttttttttttttttttttttttt

SEQ ID NO: 5 - DNA, 20 bp, Artificial Sequence
  Synthetic Polynucleotide
  misc_feature (20)..(20): Modified with /iSp18//iSp18//branch//Stearyl// Stearyl/
tccatgagcttcctgagctt

SEQ ID NO: 6 - DNA, 20 bp, Artificial Sequence
  Synthetic Polynucleotide
  misc_feature (1)..(20): Modified with a phosphorothioate linkage
  misc_feature (20)..(20): Modified with /iSp18//iSp18//branch//Stearyl// Stearyl/
tccatgagcttcctgagctt

SEQ ID NO: 7 - DNA, 21 bp, Artificial Sequence
  Synthetic Polynucleotide
  misc_feature (1)..(21): Modified with a phosphorothioate linkage
  misc_feature (21)..(21): Modified with /iSp18//iSp18//Stearyl//Stearyl/
tgcttgacacctggatgggaa

SEQ ID NO: 8 - DNA, 12 bp, Artificial Sequence
  Synthetic Polynucleotide
  misc_feature (1)..(12): Modified with a phosphorothioate linkage
  misc_feature (12)..(12): Modified with /iSp18//iSp18//Stearyl//Stearyl/
cctggatgggaa

SEQ ID NO: 9 - DNA, 18 bp, Artificial Sequence
  Synthetic Polynucleotide
  misc_feature (1)..(6): Modified with 2′OMe
  misc_feature (7)..(13): Modified with a phosphorothioate linkage
  misc_feature (13)..(18): Modified with 2′OMe
  misc_feature (18)..(18): Modified with /iSp18//iSp18//Toco/
auggagcaaaacccgcag

(19) United States
(12) Patent Application Publication
(10) Pub. No.: US 2018/0042848 A1
(43) Pub. Date: Feb. 15, 2018

(54) IMMUNO-REGULATORY LIPID CONTAINING SPHERICAL NUCLEIC ACIDS

(71) Applicant: Exicure, Inc., Skokie, IL (US)

(72) Inventors: Sergei Gryaznov, San Mateo, CA (US); Aleksandar Filip Radovic-Moreno, Boston, MA (US); Christopher C. Mader, Mendham, NJ (US); Tiffany L. Halo, Mendham, NJ (US); Richard Kang, Wilmette, IL (US); Subbarao Nallagatla, Skokie, IL (US); Clayton Rische, Skokie, IL (US); Sagar Anantatatmula, Skokie, IL (US)

(73) Assignee: Exicure, Inc., Skokie, IL (US)

(21) Appl. No.: 15/552,115

(22) PCT Filed: Feb. 18, 2016
(86) PCT No.: PCT/US2016/018395

§ 371 Date: Aug. 18, 2017

Related U.S. Application Data
(60) Provisional application No. 62/117,596, filed on Feb. 18, 2015.

(51) Int. Cl. A61K 9/127; A61K 47/69; A61K 31/711; A61K 31/713

(52) U.S. Cl. CPC A61K 9/1271; A61K 31/713; A61K 47/6911; A61K 31/711

(57) ABSTRACT

Immunoregulatory spherical nucleic acids (irSNAs) composed of a lipid containing core and an inert Nucleic non-TLR antagonistic oligonucleotide shell are provided. Acid Shell These it-SNAs are useful for modulating an immune response.

### RELATED APPLICATIONS

This application claims priority under 35 U.S.C. §119(e) to U.S. Provisional Application Ser. No. 62/117,596, entitled “IMMUNO-REGULATORY LIPID CONTAINING SPHERICAL NUCLEIC ACIDS” filed on Feb. 18, 2015, which is herein incorporated by reference in its entirety.

### BACKGROUND OF THE INVENTION

Dysregulation of the immune response is a major cause of human disease. Inappropriate or excessive immune responses are observed in a variety of inflammatory disorders, sepsis, cancers, and autoimmune diseases. There are relatively few approaches that can correct aberrant or excessive pro-inflammatory signaling. Currently, most approaches used in the clinic involve the use of broad immunosuppressants, such as azathioprine (purine analog, targets dividing cells), corticosteroids (multiple targets, multiple mechanisms including reduced leukocyte migration, reduced capillary permeability, among others) cyclosporine (calcineurin inhibitor, targets T cells), or monoclonal antibodies, such as basiliximab (anti-IL2) or adalimumab (anti-TNF-alpha). While these agents have shown efficacy in a variety of disease settings, there are major limitations. Patients on azathioprine must be monitored weekly due to toxicity, including bone marrow suppression. Corticosteroids are well-known for causing a variety of adverse effects leading to increased risk of developing Cushing's syndrome, diabetes, osteoporosis, heart disease, and glaucoma, among others. Cyclosporine is nephrotoxic and can lead to renal failure. Because of their complex structure and composition, monoclonal antibody drugs show high rates of formation of drug neutralizing antibodies, which reduce or even eliminate drug efficacy over time.

### SUMMARY OF THE INVENTION

Methods and products for modulating an immune response and treating disease are provided according to the invention. In some aspects an immunoregulatory spherical nucleic acid (SNA) is provided. The SNA includes a lipid containing core and an immuno-inert oligonucleotide attached to the lipid containing core. The immuno-inert oligonucleotide forms an oligonucleotide shell. In some embodiments the oligonucleotides of the oligonucleotide shell are directly attached to the lipid containing core.

In some embodiments the oligonucleotides of the oligonucleotide shell are indirectly attached to the lipid containing core through a linker. In other embodiments the oligonucleotides of the oligonucleotide shell are indirectly attached to the lipid containing core through more than one linker. The linker may contain, for instance, one or more of the following groups: tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and derivatives thereof.

In some embodiments the oligonucleotide shell has a density of 5-1,000 oligonucleotides per SNA. In other embodiments the oligonucleotide shell has a density of 100-1,000 oligonucleotides per SNA. In yet other embodiments the oligonucleotide shell has a density of 500-1,000 oligonucleotides per SNA.

The oligonucleotides of the oligonucleotide shell may have at least one internucleoside phosphorothioate linkage. In some embodiments the oligonucleotides of the oligonucleotide shell do not have a internucleoside phosphorothioate linkage. In yet other embodiments the oligonucleotides of the oligonucleotide shell have all internucleoside phosphorothioate linkages.

The oligonucleotides of the oligonucleotide shell may have any length. In some embodiments the oligonucleotides have a length of 10 to 300 nucleotides, 10 to 100 nucleotides, 10 to 50 nucleotides, or 10 to 30 nucleotides.

In some embodiments the oligonucleotide shell is comprised of single stranded or double stranded 2′-deoxyribo-oligonucleotides. In other embodiments the oligonucleotide shell is comprised of single stranded or double stranded 2′-ribo-oligonucleotides. In yet other embodiments the oligonucleotide shell is comprised of chimeric 2′-deoxyribo-2′-ribo-oligonucleotides. The oligonucleotides of the oligonucleotide shell may have identical nucleotide sequences or it may have at least two different nucleotide sequences. In some embodiments the oligonucleotides of the oligonucleotide shell have 2-10 different nucleotide sequences.

The oligonucleotides may have the 5′ or 3′ termini exposed to the outside of the particle. In some embodiments at least 25 percent, at least 50%, at least 75% or all of the oligonucleotides have 5′ termini exposed to the outside surface of the nanostructure. In other embodiments at least 25 percent, at least 50%, at least 75% or all of the oligonucleotides have 3′ termini exposed to the outside surface of the nanostructure.

The lipid containing core in some embodiments is comprised of one or more lipids selected from: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and derivatives thereof.

In some embodiments the lipid containing core is comprised of one type of lipid. In other embodiments the lipid containing core is comprised of 2-10 different lipids.

The nanostructure in some embodiments is a self-assembling nanostructure consisting of oligonucleotide-lipid conjugates.

The SNA may further comprise an immunoinhibitory oligonucleotide associated with the SNA. The immunoinhibitory oligonucleotide may be attached to the lipid containing core or may be attached to the immune-inert oligonucleotide.

A method for modulating an immune response in a subject is provided in other aspects of the invention. The method involves administering to a subject an immunoregulatory spherical nucleic acid (SNA) as described herein in an effective amount to modulate an immune response.

In other aspects a method for treating a subject by administering to a subject having a disorder associated with an immune response an immunoregulatory spherical nucleic acid (SNA) ad described herein, in an effective amount to reduce a pro-inflammatory immune response in order to treat the disorder is provided. In some embodiments the disorder is asthma, rheumatoid arthritis, a non-alcoholic steatohepatitis, liver cirrhosis, diabetes, autoimmune disease, sepsis, atopic dermatitis, or psoriasis.

The method in some embodiments also involves administering a therapeutic protocol to the subject. The therapeutic protocol may be surgery, radiation, or a medicament.

In some embodiments the SNA is delivered by a route selected from the group consisting of oral, nasal, sublingual, intravenous, subcutaneous, mucosal, respiratory, direct injection, and dermally.

In other aspects the invention is a method for antagonizing a TLR in a cell comprising delivering the SNA as described herein to the cell. In some embodiments the TLR is selected from the group consisting of TLR 1, 2, 3, 4, 7, 8, and 9.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, Claims, and Figures. Other features, objects, and advantages of the invention will be apparent from the description and from the claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic showing an exemplary general structure of an immunoregulatory SNA (irSNA). Some of the components for achieving immuno-regulatory effects include the lipid-containing core, an oligonucleotide shell, and optionally a lipid anchor. The lipid-containing core in FIG. 1 consists of amphiphilic lipids that assemble into a small unilamellar vesicle. The oligonucleotide shell consists of nucleic acids densely arranged radially around the core. The lipid anchor consists of a hydrophobic group that enables insertion and anchoring of the nucleic acids to the lipid membrane.

FIGS. 2A-2B show that irSNAs, as a structural class, can de-activate mature human PBMC ex vivo. IrSNAs made with small unilamellar DOPC liposomes were functionalized with three different oligonucleotides: “4084F-Ext”, “4084F”, and “CTL” (Table 1). In all three cases, the irSNAs were shown to de-activate PBMCs that were matured with either (FIG. 2A) Pam3CSK4, a ligand of TLR1/2, and (FIG. 2B) LPS, a ligand of TLR4. Note that the effect could not be achieved with a bare liposome of equivalent concentration. DOPC=1,2-dioleoyl phosphatidylcholine.

FIGS. 3A-3C show the preferred structural properties for de-activating immune cells matured with Pam3CSK4 (TLR 1/2 ligand) using irSNAs. Macrophages were matured, then incubated with irSNAs containing (FIG. 3A) different backbone chemistries (PO=phosphodiester, PS=phosphorothioate), (FIG. 3B) different nucleic acid densities, and (FIG. 3C) different nucleic acid lengths (T5=5 bases, T10=10 bases, T15=15 bases, T20=20 bases, T25=25 bases). DOPC=1,2-dioleoyl phosphatidylcholine.

FIGS. 4A-4C show preferred structural properties for de-activating immune cells matured with LPS (TLR 4 ligand) using irSNAs. Macrophages were matured, then incubated with irSNAs containing (FIG. 4A) different backbone chemistries (PO=phosphodiester, PS=phosphorothioate), (FIG. 4B) different nucleic acid densities, and (FIG. 4C) different nucleic acid lengths (T5=5 bases, T10=10 bases, T15=15 bases, T20=20 bases, T25=25 bases). DOPC=1,2-dioleoyl phosphatidylcholine.

FIGS. 5A-5B show that specific de-activation of RAW-Blue cells occurs with SOPC+15% cholesterol liposome cores. RAW-Blue cells were stimulated with either (FIG. 5A) Pam3CSK4, a TLR1/2 ligand, or (FIG. 5B) LPS, a TLR4 ligand, then de-activated with the treatments indicated. The results suggest SOPC+15% Chol lipid-containing cores are able to support de-activation of immune cells.

FIGS. 6A-6B show that irSNA (irSNA) treatment leads to a reduction in ear swelling in two models of chemically-induced ear inflammation. (FIG. 6A) Oxazolone-sensitized mice were treated with a irSNA formed with Oligo 2 (SEQ ID NO: 9) 30 minutes before and 15 minutes after a secondary exposure to oxazolone. The subsequent amount of ear swelling was quantified and shown above. (FIG. 6B) The same irSNA (SEQ ID NO: 9) was used in a Phorbol-12-myristate-13-acetate (PMA) acute ear inflammation model, where ear thickness is measured as a proxy for degree of inflammation. The results show significant reduction of ear inflammation in both models by irSNA formulations but not controls. (****p<0.0001, ***p<0.001, *p<0.05 by ANOVA).

### DETAILED DESCRIPTION

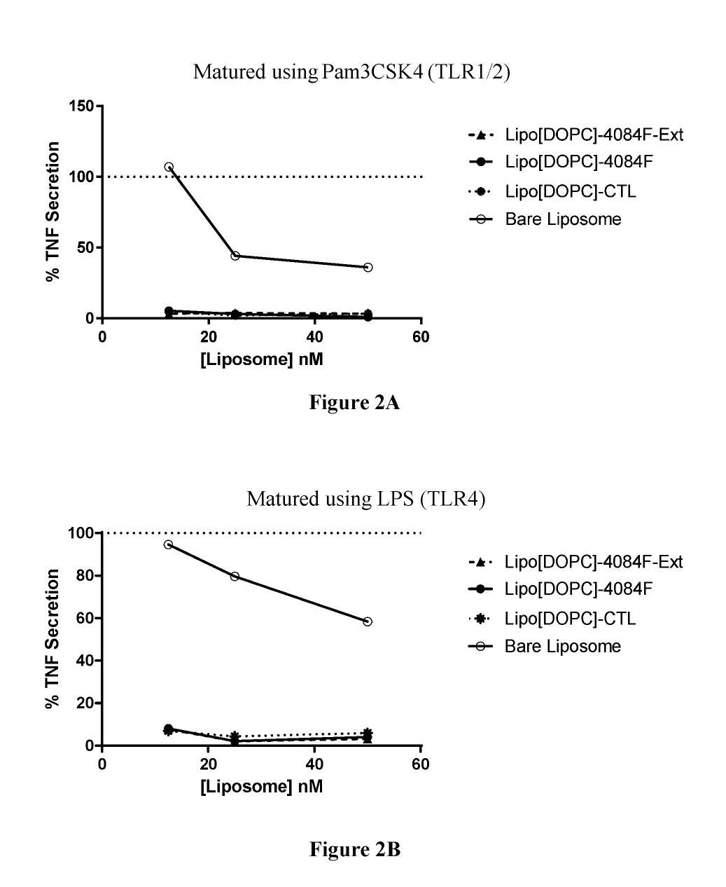

Nontoxic, biocompatible, and biodegradable lipid containing spherical nucleic acids (SNAs) that are useful for de-activating immune cells and down-regulating immune responses (“immuno-regulation”) are disclosed herein. It has been discovered, quite unexpectedly, that compounds referred to herein as immunoregulatory SNAs (irSNAs) made up of otherwise immune-inert components have potent immunoregulatory activity. It was discovered that these irSNAs composed of lipids and oligonucleotides that do not have significant immuno-modulatory properties on their own when arranged into irSNAs show potent immuno-regulatory properties both in vitro and in vivo with no evidence of toxicity. This unexpected finding, demonstrated herein both in vitro and in vivo, shows that irSNAs comprised of a variety of lipid containing cores, oligonucleotide sequences, oligonucleotide lengths, and oligonucleotide densities are capable of reducing unwanted inflammatory reactions (FIGS. 2, 3, 4, 5). Importantly, the data presented herein show that having the irSNA structure is sufficient to achieve the immuno-modulatory effects. Bare liposomes of the same composition but which lack the oligonucleotide shell do not show similar activity (FIG. 2). It appears that irSNAs, as a structural class of molecules, are uniquely able to achieve the desired reduction in activation of immune responses.

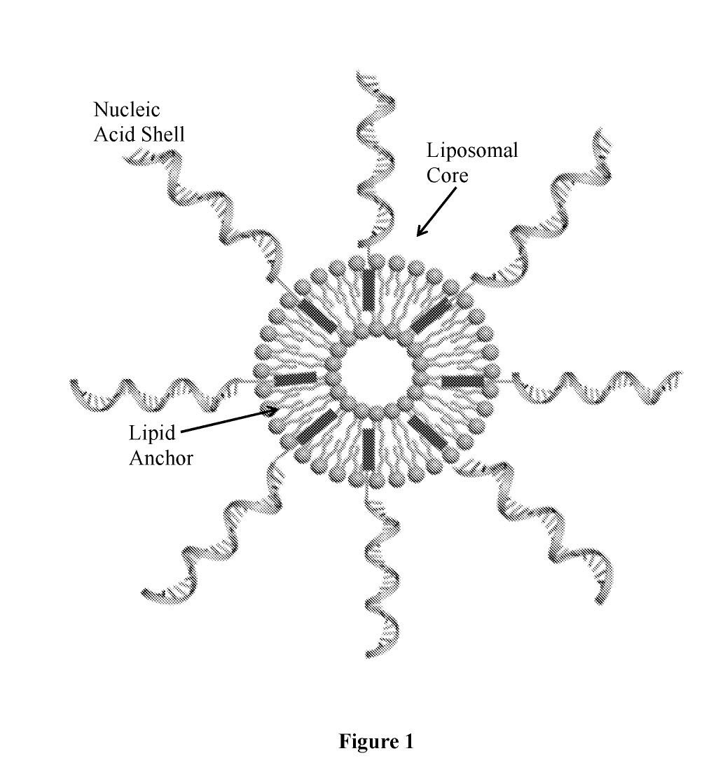

IrSNAs described herein may be useful in a wide variety of applications where there is a dysregulation of the immune response that is causing disease. In particular, the low toxicity of these structures positions them quite favorably among comparable agents, which typically have a variety of toxic side effects. Potential commercial applications include treatments for asthma, rheumatoid arthritis, non-alcoholic steatohepatitis, liver cirrhosis, diabetes, sepsis, atopic dermatitis, psoriasis, and a variety of other auto-immune disorders. IrSNAs are nanoscale constructs composed of: (1) a lipid-containing core, which is formed by arranging non-toxic carrier lipids into a small hollow structure, (2) a shell of oligonucleotides, which is formed by arranging oligonucleotides such that they point radially outwards from the core, and (3) optionally a hydrophobic (e.g. lipid) anchor group, also referred to herein as a linker, which is attached to either the 5′- or 3′-end of the oligonucleotide, depending on whether the oligonucleotides are arranged with the 5′- or 3′-end facing outward from the core (FIG. 1). The anchor acts to drive insertion into the liposome and to anchor the oligonucleotides to the lipid-containing core.

The lipid-containing core can be constructed from a wide variety of lipids known to those in the art including but not limited to: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

The exterior of the lipid-containing core has an oligonucleotide shell. The oligonucleotide shell can be constructed from a wide variety of nucleic acids including, but not limited to: single-stranded deoxyribonucleotides, ribonucleotides, and other single-stranded oligonucleotides incorporating one or a multiplicity of modifications known to those in the art, double-stranded deoxyribonucleotides, ribonucleotides, and other double-stranded oligonucleotides incorporating one or a multiplicity of modifications known to those in the art, oligonucleotide triplexes incorporating deoxyribonucleotides, ribonucleotides, or oligonucleotides that incorporate one or a multiplicity of modifications known to those in the art. In this particular invention, the L-SNAs described herein are constructed from oligonucleotides that do not have significant immune-modulating ability on their own, in contrast to formulations where the unstructured, unformulated oligonucleotides can be shown to have significant and potent immune-modulating ability. These oligonucleotides are referred to herein as immune-inert oligonucleotides. Thus, an immuno-inert oligonucleotide as used herein is an oligonucleotide which fails to have an effect on an immune response, relative to an immunostimulatory or immunoinhibitory oligonucleotide. Immunostimulatory and immunoinhibitory oligonucleotides are known in the art. For instance these oligonucleotides are disclosed in PCT Patent applications PCT/US2014/048291 and PCT/US2014/048294, each of which is incorporated by reference.

In some embodiments, the oligonucleotide shell is attached to the surface of the lipid-containing core through conjugation to one or a multiplicity of linker molecules including but not limited to: tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis).

An oligonucleotide of the irSNA and optionally attached to a lipid containing core can be single stranded or double stranded. A double stranded oligonucleotide is also referred to herein as a duplex. Double-stranded oligonucleotides of the invention can comprise two separate complementary nucleic acid strands.

As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded oligonucleotide can be double-stranded over its entire length, meaning it has no overhanging single-stranded sequences and is thus blunt-ended. In other embodiments, the two strands of the double-stranded oligonucleotide can have different lengths producing one or more single-stranded overhangs. A double-stranded oligonucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded oligonucleotide of the invention contains at least or up to 1, 2, 3, 4, 5, 6, 7, 8, 9, 10, 11, 12, 13, 14, or 15 mismatches.

Oligonucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, oligonucleotides associated with the invention may only contain modified or unmodified sugar moieties, while in other instances, oligonucleotides contain some sugar moieties that are modified and some that are not.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′—OH group replaced by an H, alkoxy (or OR), R or alkyl, halogen, SH, SR, amino (such as NH₂, NHR, NR₂,), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. The sugar moiety can also be a hexose.

The term “hydrophobic modifications’ refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; and naphthyl.

The term “heteroatom” includes atoms of any element other than carbon or hydrogen. In some embodiments, preferred heteroatoms are nitrogen, oxygen, sulfur and phosphorus. The term “hydroxy” or “hydroxyl” includes groups with an —OH or —O⁻ (with an appropriate counterion). The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)₀₋₃NR′R″, (CR′R″)₀₋₃CN, NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, or (CR′R″)₀₋₃OR′ groups; wherein each R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

In some aspects, the nucleomonomers of an oligonucleotide of the invention are RNA nucleotides, including modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The term “nucleotide” includes nucleosides which further comprise a phosphate group or a phosphate analog.

As used herein, the term “linkage” includes a naturally occurring, unmodified phosphodiester moiety (—O—(PO²⁻)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothioate linkages.

In some aspects, oligonucleotides of the invention comprise 3′ and 5′ termini (except for circular oligonucleotides). The 3′ and 5′ termini of an oligonucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

In some aspects, oligonucleotides can comprise both DNA and RNA.

In some aspects, at least a portion of the contiguous oligonucleotides are linked by a substitute linkage, e.g., a phosphorothioate linkage. The presence of substitute linkages can improve pharmacokinetics due to their higher affinity for serum proteins.

The oligonucleotides of the irSNA are preferably in the range of 6 to 100 bases in length. However, nucleic acids of any size greater than 6 nucleotides (even many kb long) are useful. Preferably the nucleic acid is in the range of between 8 and 100 and in some embodiments between 8 and 50, 8 and 40, 8 and 30, 6 and 50, 6 and 40, or 6 and 30 nucleotides in size.

The surface density of the oligonucleotides may depend on the size and type of the core and on the length, sequence and concentration of the oligonucleotides. A surface density adequate to make the nanoparticles stable and the conditions necessary to obtain it for a desired combination of nanoparticles and oligonucleotides can be determined empirically. Generally, a surface density of at least 100 oligonucleotides per particle will be adequate to provide stable core-oligonucleotide conjugates. Preferably, the surface density is at least 200, 300, 400, 500, 600, 700, 800, 900, 1,000, 1,200, 1,400, 1,600, 1,800, or 2,000 oligonucleotides per particle.

Aspects of the invention relate to delivery of irSNAs to a subject for therapeutic and/or diagnostic use. The SNAs may be administered alone or in any appropriate pharmaceutical carrier, such as a liquid, for example saline, or a powder, for administration in vivo. They can also be co-delivered with larger carrier particles or within administration devices. The SNAs may be formulated. The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments, irSNAs associated with the invention are mixed with a substance such as a lotion (for example, aquaphor) and are administered to the skin of a subject, whereby the irSNAs are delivered through the skin of the subject. It should be appreciated that any method of delivery of nanoparticles known in the art may be compatible with aspects of the invention.

For use in therapy, an effective amount of the SNAs can be administered to a subject by any mode that delivers the SNAs to the desired cell. Administering pharmaceutical compositions may be accomplished by any means known to the skilled artisan. Routes of administration include but are not limited to oral, parenteral, intramuscular, intravenous, subcutaneous, mucosal, intranasal, sublingual, intratracheal, inhalation, ocular, vaginal, dermal, rectal, and by direct injection.

Thus, the invention in one aspect involves the finding that immune-inert oligonucleotides are highly effective in mediating immune modulatory effects. These oligonucleotides are useful therapeutically and prophylactically for modulating the immune system to treat allergy, asthma, autoimmune disease, and other inflammatory based diseases.

According to other aspects the invention is a method of treating a subject, involving administering to the subject the irSNA as described herein in an effective amount to reduce an immune response. In some embodiments the subject has an autoimmune disease, asthma, an allergic disease, or an inflammatory disease, or is a candidate for or the recipient of tissue or organ transplant.

A subject shall mean a human or vertebrate animal including but not limited to a dog, cat, horse, cow, pig, sheep, goat, turkey, chicken, primate, e.g., monkey, and fish (aquaculture species), e.g. salmon. Thus, the invention can also be used to treat cancer and tumors, infections, and allergy/asthma in non-human subjects.

As used herein, the term treat, treated, or treating when used with respect to an disorder such as an inflammatory disease, autoimmune disease, allergy, or asthma refers to a prophylactic treatment which increases the resistance of a subject to development of the disease (e.g., to inflammation) or, in other words, decreases the likelihood that the subject will develop the disease as well as a treatment after the subject has developed the disease in order to fight the disease (e.g., reduce or eliminate the inflammation) or prevent the disease from becoming worse.

A subject having an allergy is a subject that has or is at risk of developing an allergic reaction in response to an allergen. An allergy refers to acquired hypersensitivity to a substance (allergen). Allergic conditions include but are not limited to eczema, allergic rhinitis or coryza, hay fever, conjunctivitis, bronchial asthma, urticaria (hives) and food allergies, and other atopic conditions.

The irSNAs are also useful for treating and preventing autoimmune disease. Autoimmune disease is a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

A “self-antigen” as used herein refers to an antigen of a normal host tissue. Normal host tissue does not include cancer cells. Thus an immune response mounted against a self-antigen, in the context of an autoimmune disease, is an undesirable immune response and contributes to destruction and damage of normal tissue, whereas an immune response mounted against a cancer antigen is a desirable immune response and contributes to the destruction of the tumor or cancer. Thus, in some aspects of the invention aimed at treating autoimmune disorders it is not recommended that the immunoregulatory nucleic acids be administered with self-antigens, particularly those that are the targets of the autoimmune disorder.

In other instances, the immunoregulatory nucleic acids may be delivered with low doses of self-antigens. A number of animal studies have demonstrated that mucosal administration of low doses of antigen can result in a state of immune hyporesponsiveness or “tolerance.” The active mechanism appears to be a cytokine-mediated immune deviation away from a Th1 towards a predominantly Th2 and Th3 (i.e., TGF-β dominated) response. The active suppression with low dose antigen delivery can also suppress an unrelated immune response (bystander suppression) which is of considerable interest in the therapy of autoimmune diseases, for example, rheumatoid arthritis and SLE. Bystander suppression involves the secretion of Th1-counter-regulatory, suppressor cytokines in the local environment where proinflammatory and Th1 cytokines are released in either an antigen-specific or antigen-nonspecific manner. “Tolerance” as used herein is used to refer to this phenomenon. Indeed, oral tolerance has been effective in the treatment of a number of autoimmune diseases in animals including: experimental autoimmune encephalomyelitis (EAE), experimental autoimmune myasthenia gravis, collagen-induced arthritis (CIA), and insulin-dependent diabetes mellitus. In these models, the prevention and suppression of autoimmune disease is associated with a shift in antigen-specific humoral and cellular responses from a Th1 to Th2/Th3 response.

In another aspect, the present invention is directed to a kit including one or more of the compositions previously discussed. A “kit,” as used herein, typically defines a package or an assembly including one or more of the compositions of the invention, and/or other compositions associated with the invention, for example, as previously described. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions that may be associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

In some embodiments, a kit associated with the invention includes one or more lipid cores. A kit can also include one or more oligonucleotides. A kit can also include one or more anchors or linkers.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the use of the compositions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoting” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

All references, including patent documents, disclosed herein are incorporated by reference in their entirety.

In order that the invention described herein may be more fully understood, the following examples are set forth. The examples described in this application are offered to illustrate the compounds, pharmaceutical compositions, and methods provided herein and are not to be construed in any way as limiting their scope.

### EQUIVALENTS

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### EXAMPLES

### Example 1

A panel of oligonucleotides (sequences shown in Table 1) were 3′-modified with a di-stearyl group and incorporated into small unilamellar vesicles (<50 nm in diameter) composed of 1,2-dioleoyl-sn-glycero-3-phosphatidylcholine (DOPC) (FIG. 1) at a variety of oligonucleotide densities. These lipid containing SNAs were tested for their ability to down-regulate immune cells that are activated with two representative TLR ligands: Pam3CSK4 (TLR 1/2) and LPS (TLR 4). These TLR ligands are implicated in a variety of diseases. Importantly, there was no component in the lipid containing SNA formulations that when administered in isolation would be expected to de-activate immune cells activated with these ligands. The ability to de-activate immune cells matured with these two ligands in this manner was quite unexpected.

### Methods

### Lipid Containing SNA Synthesis

200 mg of 1,2-dioleoyl-sn-glycero-3-phosphatidylcholine (DOPC) were dissolved in 4 mL dichloromethane (DCM) in a glass container. The lipids were then dried onto the walls of the glass container in a thin film by gently drying under argon until all solvent had evaporated. Any residual solvent was removed by overnight lyophilization. The next day, the lipids were reconstituted in 10 mL of liposome buffer (150 mM NaCl, 20 mM HEPES) by vortex and sonication, then passed through 2-5 freeze thaw cycles prior to serial extrusion through 100 nm, 50 nm, then 30 nm extrusion membranes. Following extrusion oligonucleotide based SNA particles were generated for each of the oligonucleotides shown in Table 1. For each SNA 1-2 μmol of oligonucleotide was mixed with the liposome preparation and incubated overnight at 4° C. to form the lipid containing SNAs. The following day, the lipid containing SNAs were purified by tangential flow filtration using a 300 kDa membrane cutoff filter using >5 volume exchanges of 1×PBS.

### In Vitro Testing

RAW-Blue cells (InVivoGen), a reporter murine macrophage cell line derived from RAW 264.7 cells containing a NF-kB inducible secreted alkaline phosphatase (SEAP) were seeded at 10⁵ cells/well in a 96-well plate. In some experiments, human peripheral blood mononuclear cells (PBMC) were used, and these were seeded at a density of <10⁶/well. Cells were matured by incubation with an appropriate concentration of Pam3CSK4 or LPS for a period of at least 2 hours. Following this incubation, the indicated quantities of irSNAs were added on top of the existing agonist-containing media (without washing steps) and then incubated overnight at 37° C. and 5% CO₂ in a humidified chamber. The following day, the supernatants were probed for either SEAP activity using the QuantiBlue reagent (InVivoGen) following the manufacturer recommended protocol (RAW-Blue cells), or probed for levels of human TNF-alpha using TNF-specific ELISA kit.

### In Vivo Testing

For studies involving the use of oxazolone in inducing ear swelling, mice were randomized into groups of 8 each and sensitized with a primary oxazolone exposure (100 μL, 1% in acetone) by topical application to their shaved abdominal surface. Seven days after sensitization, formulated test substances, vehicle, or reference positive control compound were topically applied on the right ear surface 30 minutes before and then 15 minutes after sensitization with oxazolone (20 μL, 0.5% in acetone). Ear swelling was measured 24 hours after the second oxazolone application in all treatment groups.

For studies involving the use of phorbol 12-myristate-13-acetate (PMA), male ICR mice (BioLasco Taiwan & Charles River Laboratories) were randomized into groups of 8 mice per group. Test substances, vehicle, and positive control reference compound were applied 30 minutes before and 15 minutes after PMA challenge. Ear swelling was measured 6 hours after PMA application.

### Results

Lipid Containing SNAs Including Immune-Inert Oligonucleotides (Also Referred to Herein as Immunoregulatory Spherical Nucleic Acids (irSNAs)), Unexpectedly De-Activate Peripheral Blood Mononuclear Cells Matured with TLR 1, 2, and 4 Ligands Ex Vivo

The ability of irSNAs containing three different sequences to de-activate PBMCs that had been previously matured with Pam3CSK4 (ligand of TLR 1/2) and LPS (ligand of TLR 4) was evaluated. These TLR ligands were selected because they are potent activators of immune cells and because there is no component of the irSNA that would be expected to de-activate cells activated by these ligands in isolation (i.e. not in irSNA formulation). The data show that all three types of irSNAs are able to reduce secretion of TNF by PBMCs at all doses tested consistent with immune cell de-activation, whereas a bare liposome that was used to construct the SNA showed little significant activity (FIG. 2). The 4084F-Ext sequence has been shown to reduce immune cell activation by TLR 7/8/9 ligands, but is not known to inhibit activation by TLR 1/2/4 ligands. Similarly, the 4084F sequence is known to reduce immune cell activation by TLR 9 ligands, but not TLR 1/2/4 ligands. The sequence here labeled CTL is not known to inhibit activation by any immune activating ligand. In all cases, the irSNAs significantly inhibit activation of PBMCs by TLR 1/2 (FIG. 2A) and TLR 4 ligands (FIG. 2B). Importantly, this effect appeared to be a broad phenomenon that was observed independent of nucleic acid sequence. We conclude that the de-activation of PBMCs stimulated with TLR 1, 2, and 4 ligands appears to be uniquely achieved by the irSNA assembly, and not by any single specific component that is delivered by the irSNA.

High Oligonucleotide Density and Other Structural Changes to irSNAs Increase the Potency of Immune Cell De-Activation

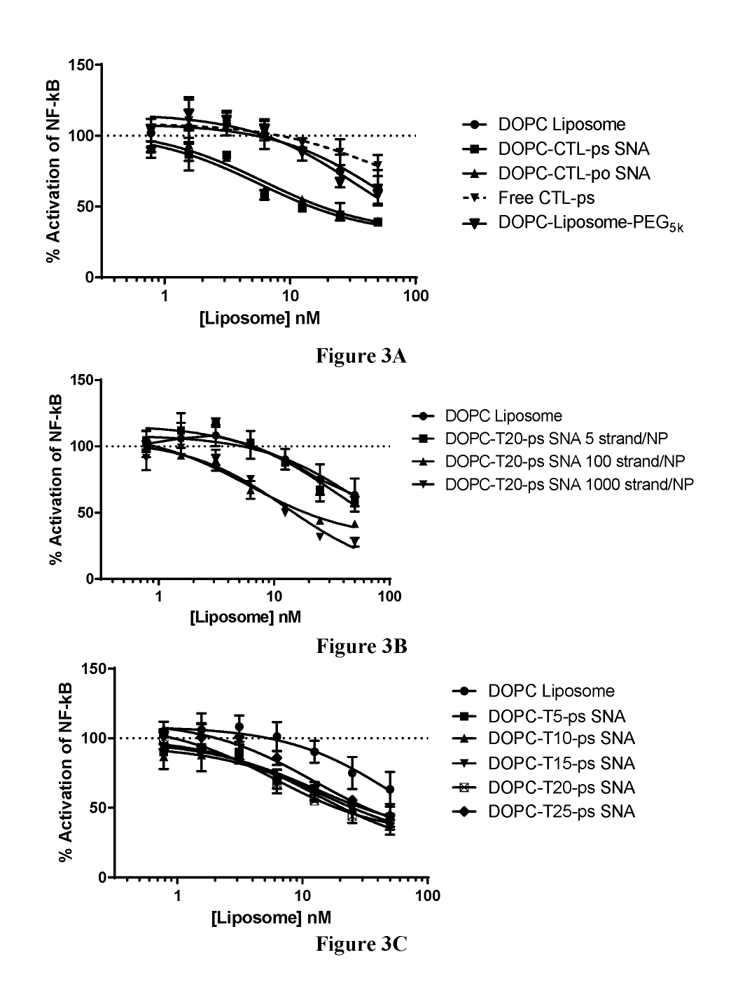

We sought to evaluate the specific structural requirements that modulate the ability of irSNAs to de-activate mature immune cells matured with Pam3CSK4 and LPS. This has important implications in designing optimal immune cell de-activating irSNAs. We first varied the internucleotide linkage chemistry in Pam3CSK4 matured RAW Blue cells (FIG. 3A) to test the impact of a phosphodiester (PO) vs phosphorothioate (PS) backbone. The results show that for Pam3CSK4-activated cells, the results did not appear to depend on the backbone, as results were similar for both PO or PS irSNAs. Importantly, we again note that an oligonucleotide that was not pre-formulated into a liposome showed greatly reduced activity, as did DOPC liposomes surface modified with PEG-5000, indicating that the effect was specific to the irSNA architecture. Next, we sought to evaluate the effect of oligonucleotide density on activity. We tested formulations containing 5, 100, or 1000 strands/nanoparticle (NP). The results show that oligonucleotide density has an impact on activity. irSNAs that had a density of 100 or 1000 strands/NP demonstrated significantly improved activity as compared to an irSNA loaded with 5 strands/NP (FIG. 3B). In contrast, the low density irSNAs as tested in this example showed activity that was comparable to that achieved by a bare DOPC liposome (FIG. 3B). thus, in some embodiments, preferred irSNA are loaded with at least 5 strands/NP. Finally, we sought to evaluate the impact of oligonucleotide length on activity, comparing irSNAs with oligonucleotides that were 5, 10, 15, 20, and 25 bases long (FIG. 3C). The results show that for cells matured with Pam3CSK4, there appears to be relatively little impact of oligonucleotide length. In summary, high oligonucleotide density appears to be a major parameter that yields optimal irSNA for the constructs tested for de-activating immune cells matured with TLR 1/2 ligands, such as Pam3CSK4.

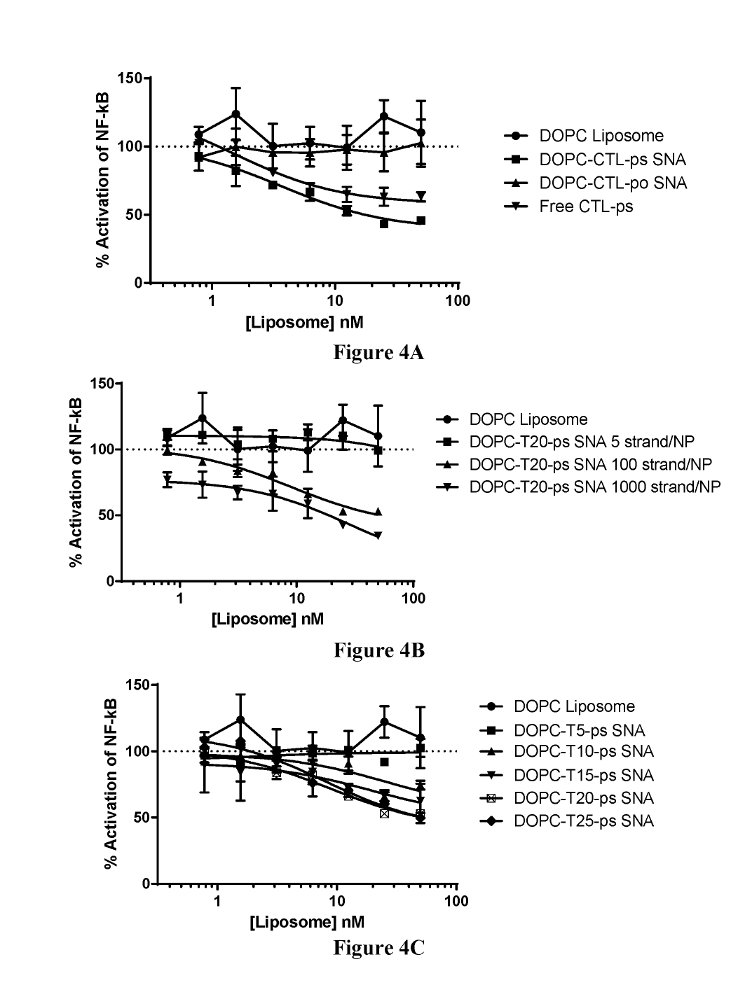

We repeated a similar set of experiments that led to FIG. 3 with RAW Blue cells that were matured with LPS (TLR4 ligand) to assess the general applicability across multiple TLR agonist-treated cells. The results show that for LPS-matured RAW Blue cells, the oligonucleotide backbone chemistry of the studied constructs appeared to play a role, with phosphorothioate linkages leading to significantly greater activity (FIG. 4A). Similar to cells pre-treated with Pam3CSK4, cells treated with LPS demonstrate de-activation depending on the density of the irSNA treatment, with higher density generally showing improved activity (FIG. 4B). We also found that for LPS-matured cells, activity appeared to depend significantly on oligonucleotide length (FIG. 4C). Similar to results shown in Pam3CSK4 cells, oligonucleotides that are 5 bases in length show little activity. A length of 10 bases showed an effect, and as length is increased, activity was improved up to 20 bases. There did not appear to be significant benefit from improving the oligonucleotide length to 25 bases, though no loss in activity was observed. In summary, high oligonucleotide density and phosphorothioate backbone are parameters that can be adjusted for achieving optimal deactivaton of immune cells matured with TLR4 ligands, such as LPS.

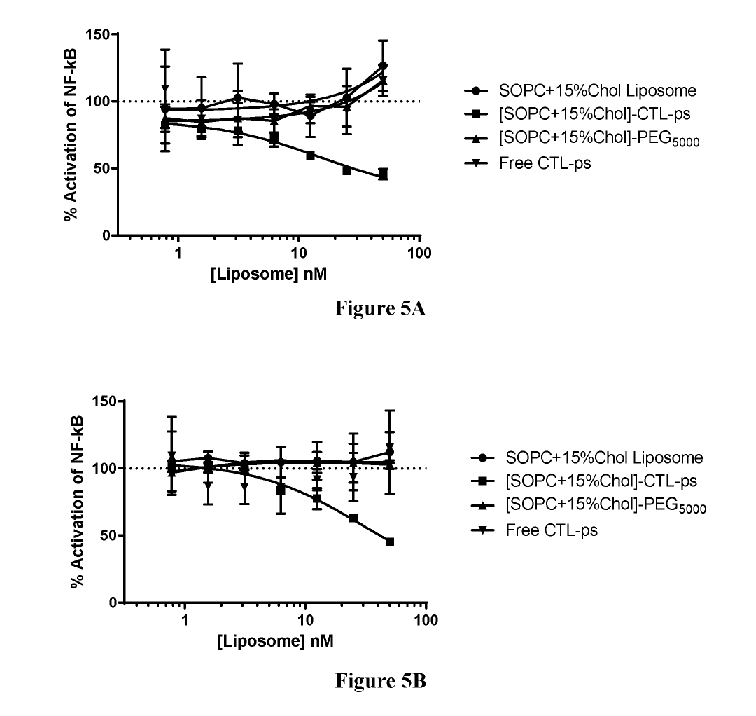

Finally, we assessed whether the effects were restricted to DOPC irSNAs, or if different lipid-containing cores could be used. To answer this question, we formed bare liposomes made with SOPC+15% cholesterol, liposomes with SOPC+15% cholesterol cores decorated with PEG-5000, and irSNAs from lipid-containing cores made with SOPC+15% cholesterol using the CTL-ps sequence (Table 1). The results show that, similar to DOPC irSNAs, the SOPC+15% cholesterol SNAs de-activated immune cells in a manner that critically depended on the presence of oligonucleotide and could not be achieved by bare liposomes or by PEGylated liposomes (FIG. 5). We conclude that the specific type of lipid-containing nanoparticle core is not essential to achieving immune cell de-activation, as similar results could be achieved with different lipid-containing cores (DOPC and SOPC+15% cholesterol).

Taken together, these results suggest that irSNAs are able to de-activate immune cells significantly better than bare liposomes, free oligonucleotide, or PEGylated liposomes, suggesting that irSNAs, as a structural class, have the ability to de-activate mature immune cells through an as yet undefined mechanism. To achieve optimal effects, increasing the density of oligonucleotide on the irSNAs (to 1000 oligos/SNA) and using phosphorothioate backbone modifications yielded the most potent and broadly applicable immune cell-deactivating effects.

TABLE 1
Nucleic acid sequences tested

| | SEQ | |
|---|---|---|
| Name | ID NO: | Sequence (5′-3′) |
| T5-ps | | T*T*T*T*T*/iSp18//iSp18//branch// |
| | | Stearyl//Stearyl/ |
| T10-ps | 1 | T*T*T*T*T*T*T*T*T*T*/iSp18// |
| | | iSp18//branch//Stearyl//Stearyl/ |
| T15-ps | 2 | T*T*T*T*T*T*T*T*T*T*T*T*T*T*T*/ |
| | | iSp18//iSp18//branch//Stearyl// |
| | | Stearyl/ |
| T20-ps | 3 | T*T*T*T*T*T*T*T*T*T*T*T*T*T*T*T* |
| | | T*T*T*T*/iSp18//iSp18//branch// |
| | | Stearyl//Stearyl/ |
| T25-ps | 4 | T*T*T*T*T*T*T*T*T*T*T*T*T*T*T*T* |
| | | T*T*T*T*T*T*T*T*T*/iSp18//iSp18// |
| | | branch//Stearyl//Stearyl/ |
| CTL-po | 5 | TCCATGAGCTTCCTGAGCTT/iSp18// |
| | | iSp18//branch//Stearyl//Stearyl/ |
| CTL-ps | 6 | T*C*C*A*T*G*A*G*C*T*T*C*C*T*G*A* |
| | | G*C*T*T*/iSp18//iSp18//branch// |
| | | Stearyl//Stearyl/ |
| 4084F- | 7 | T*G*C*T*T*G*A*C*A*C*C*T*G*G*A*T* |
| Ext | | G*G*G*A*A/iSp18//iSp18//Stearyl// |
| | | Stearyl/ |
| 4084F | 8 | C*C*T*G*G*A*T*G*G*G*A*A/iSp18// |
| | | iSp18//Stearyl//Stearyl/ |
| Oligo | 9 | mAmUmGmGmAmGC*A*A*A*A*C*mCmCmGmC |
| 2 | | mAmG/iSp18//iSp18//Toco/ |

iSp18 = internal spacer 18 (6 ethylene glycol repeats),
Toco = tocopherol.
mN indicates 2′OMe RNA base,
*indicates phosphorothioate linkage.

irSNA Treatment Reduces Ear Swelling in Two Models of Chemically-Induced Ear Inflammation

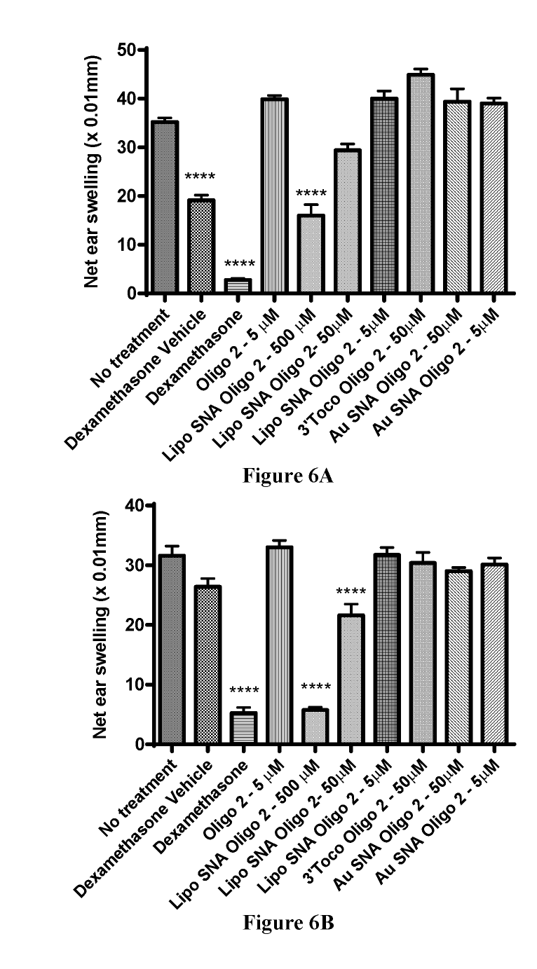

irSNAs formed with an immune-inert oligonucleotide (not expected to have biological activity) was tested for its ability to reduce ear swelling in models of chemically-induced ear inflammation (Table 1). Oxazolone is known to induce a delayed-type hypersensitivity (DTH) reaction in mice and has been used extensively as a model of human diseases of the skin, including atopic dermatitis, eczema, and psoriasis. In this model, mice are first sensitized by abdominal application of oxazolone, which primes a DTH response. A secondary exposure to the ear yields a localized inflammatory response that can be easily quantified by measuring ear thickness. We hypothesized that, based on the in vitro and ex vivo results, irSNAs could dampen the DTH response to oxazolone. To test this hypothesis, irSNAs or controls were applied to the ear of oxazolone-sensitized mice 30 minutes before and then 15 minutes after the secondary challenge with oxazolone. The results show that irSNAs (Oligo 2, SEQ ID NO: 9, Table 1) were able to induce a significant reduction in ear thickness (FIG. 6A, p<0.0001). Interestingly, the same oligonucleotides not formulated into irSNAs (3′-Toco Oligo 2) or formulated onto 13 nm gold cores (Au-SNA Oligo 2) did not show similar effects (FIG. 6a, p>0.05), showing that the results are specific to the irSNA structure.

Similarly, we sought to assess whether irSNAs are able to reduce inflammation in a model of acute inflammation. Phorbol 12-myristate 13-acetate (PMA) induces a localized Th1 inflammatory reaction leading to increased vascular permeability, a leukocyte infiltrate, and increased secretion of various pro-inflammatory cytokines. In this model, mice were treated with irSNA constructs or control 30 minutes before and 15 minutes after PMA addition. The resulting ear thickness changes were measured 6 hours later. The results show that, similar to the oxazolone-induced DTH model, the irSNAs, but not 3′-Toco oligos or Au-SNAs, show a significant reduction in ear thickness (FIG. 6B, p<0.0001).

In summary, we conclude that irSNAs, as a structural class, unexpectedly appear to de-activate immune cells in vitro and ex vivo, and reduce inflammation in animal models of topical DTH and acute inflammation. The same effects were not observed using bare liposomes, free oligonucleotides, or gold core SNAs at similar concentrations, suggesting that the effects appear to be uniquely suited to a lipid containing core. The potency of irSNAs may be modulated by factors including oligonucleotide backbone chemistry, oligonucleotide density, and oligonucleotide length, depending on the type of activating ligand used to mature the cells, but optimal formulations for the tested combinations generally contained high oligonucleotide density (1000 strands/SNA) and phosphorothioate oligonucleotide backbones. Incorporating components into the irSNAs that activate immune cells with high potency can overcome the immune-regulatory activity disclosed in this application. Taken together, these results suggest that irSNAs have applications in a wide variety of disease applications where down-regulation of immune responses may have therapeutic benefit, including asthma, rheumatoid arthritis, non-alcoholic steatohepatitis, liver cirrhosis, diabetes, sepsis, atopic dermatitis, psoriasis and a variety of other auto-immune or atopic disorders.

### REFERENCES

1. Abdollahi-Roodsaz, S., et al., Inhibition of Toll-like receptor 4 breaks the inflammatory loop in autoimmune destructive arthritis. Arthritis Rheum, 2007. 56(9): p. 2957-67.
2. Chun, E., et al., Toll-like receptor expression on peripheral blood mononuclear cells in asthmatics; implications for asthma management. J Clin Immunol, 2010. 30(3): p. 459-64.
3. Fukata, M., et al., Constitutive activation of epithelial TLR4 augments inflammatory responses to mucosal injury and drives colitis-associated tumorigenesis. Inflamm Bowel Dis, 2011. 17(7): p. 1464-73.
4. Gambuzza, M. E., et al., Toll-Like Receptors in Alzheimer's Disease: a Therapeutic Perspective. CNS Neurol Disord Drug Targets, 2014.
5. Kandimalla, E. R., et al., Design, synthesis and biological evaluation of novel antagonist compounds of Toll-like receptors 7, 8 and 9. Nucleic Acids Res, 2013. 41(6): p. 3947-61.
6. Monaco, C., et al., Toll-like receptor-2 mediates inflammation and matrix degradation in human atherosclerosis. Circulation, 2009. 120(24): p. 2462-9.
7. Robbins, M., et al., 2′-O-methyl-modified RNAs act as TLR7 antagonists. Mol Ther, 2007. 15(9): p. 1663-9.
8. Suarez-Farinas, M., et al., Suppression of molecular inflammatory pathways by Toll-like receptor 7, 8, and 9 antagonists in a model of IL-23-induced skin inflammation. PLoS One, 2013. 8(12): p. e84634.
9. Wang, D., et al., Oligodeoxyribonucleotide-based antagonists for Toll-like receptors 7 and 9. J Med Chem, 2009. 52(2): p. 551-8.
10. Wang, D., et al., Oligodeoxyribonucleotide-based antagonists for Toll-like receptors 7 and 9. J Med Chem, 2009. 52(2): p. 551-8.
11. Yu, D., et al., Modifications incorporated in CpG motifs of oligodeoxynucleotides lead to antagonist activity of toll-like receptors 7 and 9. J Med Chem, 2009. 52(16): p. 5108-14.
12. Zhang, Y., et al., TLR9 blockade inhibits activation of diabetogenic CD8+ T cells and delays autoimmune diabetes. J Immunol, 2010. 184(10): p. 5645-53.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An immunoregulatory spherical nucleic acid (SNA), comprising
a lipid bilayer containing core, wherein an immuno-inert oligonucleotide, which is not a toll-like receptor (TLR) antagonist, is attached to the lipid bilayer containing core and thus forms an oligonucleotide shell.

2. The SNA of claim 1, wherein the oligonucleotides of the oligonucleotide shell are directly attached to the lipid containing core.

3. The SNA of claim 1, wherein the oligonucleotides of the oligonucleotide shell are indirectly attached to the lipid containing core through a linker, which is a lipid anchor group.

4. The SNA of claim 1, wherein the oligonucleotides of the oligonucleotide shell are indirectly attached to the lipid containing core through more than one linker, which are lipid anchor groups.

5. (canceled)

6. The SNA of claim 1, wherein the oligonucleotide shell has a density of 5-1,000 oligonucleotides per SNA.

7.-11. (canceled)

12. The SNA of claim 1, wherein the oligonucleotides of the oligonucleotide shell have a length of 10 to 100 nucleotides.

13.-15. (canceled)

16. The SNA of claim 1, wherein the oligonucleotide shell is comprised of single stranded or double stranded 2′-deoxyribo-oligonucleotides.

17. The SNA of claim 1, wherein the oligonucleotide shell is comprised of single stranded or double stranded 2′-ribo-oligonucleotides.

18. The SNA of claim 1, wherein the oligonucleotide shell is comprised of chimeric 2′-deoxyribo-2′-ribo-oligonucleotides.

19. (canceled)

20. (canceled)

21. The SNA of claim 1, wherein the oligonucleotides of the oligonucleotide shell have 2-10 different nucleotide sequences.

22. The SNA of claim 1, wherein at least 25 percent of the oligonucleotides have 5′-termini exposed to the outside surface of the nanostructure.

23. The SNA of claim 1, wherein all of the oligonucleotides have 5′ termini exposed to the outside surface of the nanostructure.

24. The SNA of claim 1, wherein at least 25 percent of the oligonucleotides have 3′-termini exposed to the outside surface of the nanostructure.

25. The SNA of claim 1, wherein all of the oligonucleotides have 3′-termini exposed to the outside surface of the nanostructure.

26. (canceled)

27. The SNA of claim 1, wherein the lipid bilayer containing core is comprised of one type of lipid.

28. The SNA of claim 1, wherein the lipid bilayer containing core is comprised of 2-10 different lipids.

29. A method for modulating an immune response in a subject, comprising
administering to a subject an immunoregulatory spherical nucleic acid (SNA) of claim 1, in an effective amount to modulate an immune response in the subject.

30. A method for treating a subject, comprising
administering to a subject having a disorder associated with an immune response an immunoregulatory spherical nucleic acid (SNA) of claim 1, in an effective amount to reduce a pro-inflammatory immune response in the subject in order to treat the disorder.

31.-39. (canceled)

40. The method of claim 30, further comprising administering a therapeutic protocol to the subject.

41.-44. (canceled)

45. A method for antagonizing activity of TLRs in a cell comprising delivering the SNA of claim 1 to the cell.

46. (canceled)
